(19) United States
(12) Patent Application Publication
(10) Pub. No.: US 2007/0082042 A1
(43) Pub. Date: Apr. 12, 2007

(54) Multiple-layered liposome and preparation method thereof

(76) Inventors: Deok-Hoon Park, Kyunggi-Do (KR); Jong-Sung Jung, Kyunggi-Do (KR)

Correspondence Address: ANDERSON, KILL & OLICK, P.C., 1251 AVENUE OF THE AMERICAS, NEW YORK,, NY, 10020-1182, US

(21) Appl. No.: 10/579,163

(22) PCT Filed: Aug. 6, 2004
(86) PCT No.: PCT/KR04/01989

§ 371 Date: May 11, 2006

(51) Int. Cl. A61K 9/127

(52) U.S. Cl. 424/450

(57) ABSTRACT

Disclosed are multilayered liposomes for transdermal absorption and a method of preparing the liposomes. The multilayered liposomes are prepared using a mixture of oil-phase components comprising squalane, sterols, ceramides, neutral lipids or oils, fatty acids and lecithins, is 200 to 5000 nm in particle size, and is capable of entrapping a physiologically active substance. The multilayered liposomes entrap a larger amount of a physiologically active substance and are structurally stable when encapsulating the physiologically active substance, compared to unilamellar liposomes. Also, they are prepared by a simple and cost-effective process not using a high-pressure homogenizer but using a general homo mixer. Further, since the multilayered liposomes are prepared in a larger size than the intercellular spaces in the stratum corneum, they overcome the tension of surrounding cells when passing through the intercellular spaces and are thus able to penetrate into the dermal layer, compared to nano-sized unilamellar liposomes. Thus, the multilayered liposomes are useful for enhancing the transdermal absorption of physiologically active substances.

### TECHNICAL FIELD

The present invention relates to a multilayered liposome for transdermal absorption. More particularly, the present invention relates to a multilayered liposome for transdermal absorption which is capable of entrapping a physiologically active substance, wherein the liposome is prepared using a mixture of oil-phase components comprising squalane, sterols, ceramides, neutral lipids or oils, fatty acids and lecithins and is 200 to 5000 nm in particle size, and a method of preparing the liposome.

### BACKGROUND ART

When materials known to be effective at the cellular level are applied to the skin having a complex structure, they often do not have therapeutic efficacy. For this reason, the understanding of skin structure is required for manufacturing functional cosmetics or preparing related functional raw materials. In fact, most skin research scientists and dermatologists recognize from their experience that some materials effective in vitro do not display efficacy in the body. This is basically because therapeutic efficacy is expected to be achieved only when an active ingredient arrives at skin cells due to the complex structure of the skin and the distribution pattern of skin cells in the skin.

The skin is the largest organ in the body, which is made up of three distinct layers: epidermis, dermis and subcutaneous fat. The skin serves numerous functions, and its major functions are a protective barrier function protecting the body from the outside world, regulation of body temperature, excretion, respiration, and the like. The epidermis and dermis vary in thickness according to different parts of the skin, but are generally about 2 to 5 mm in thick. With respect to the thickness of the subcutaneous fat, there is a large difference between individuals. The epidermis is the outer layer of dermis, is only about 0.1 mm thick, is mainly composed of skin cells called keratinocytes, and also contains melanocytes, which produce a brown-black pigment called melanin, and other cells involved in immune responses in the skin, all the cells being scattered among the keratinocytes. The epidermis consists of four layers: stratum basale (basal layer), stratum spinosum (spinous layer), stratum granulosum (granular layer) and stratum corneum (cornified layer). The stratum basale is the innermost cell layer. The dividing keratinocytes are located in the basal layer. The basal keratinocytes differentiate as they migrate upward through the spinous, granular and cornified layers. In the spinous layer that is above the basal layer, the cells contain bundles of keratin filaments. Above the spinous layer is the granular layer, in which the cells are filled with granules when observed microscopically. The cornified layer, which is the outermost layer, consists of highly keratinized dead cells in which keratin filaments are tightly bound to granules. The stratum corneum that consists of about 10 to 20 layers of fattened keratinized cells can be hydrated so that it has the ability to retain water in an amount several times its dry weight, and is about 10 μm thick. The stratum corneum acts as the main barrier when drugs for treating skin diseases or cosmetic products are applied to the skin. The intercellular spaces in the stratum corneum comprise 10 to 30% of the total volume of the stratum corneum, which are larger than those in other general tissues, and are filled with several lipid bilayers composed of neutral lipids or oils, so that they provides the barrier function to the stratum corneum. Due to this nature of the skin, particularly the barrier function of the stratum corneum, transdermal absorption of active ingredients and cosmetic agents is a very important factor when therapeutic drugs or cosmetic products are developed.

Thus, substances found to be effective in vitro should be substantially tested for permeability through the skin of animals or humans prior to application to the skin. In Korea, functional cosmetic products have limited functions of wrinkle improvement, skin whitening and ultraviolet protection. UV protection products contain chemicals capable of scattering UV light on the surface of the skin, such as titanium dioxide, or UV absorbers capable of absorbing UV light on the skin surface. Wrinkle improvement is difficult to achieve when active ingredients act in the outermost layer of the skin, that is, the stratum corneum or the epidermis. Substantial reduction in wrinkle is expected when active-ingredients act in the dermis layer which contains collagen-synthesizing cells. Also, skin whitening effects can be achieved when active ingredients act particularly on melanin-producing cells in the basal layer of the epidermis. Taken together, the potential efficacy of substances found to be efficacious in vitro in the skin of humans is closely related to their ability to penetrate the skin. Therefore, the effects of certain substances on wrinkle improvement or skin whitening depend on the substances' ability to penetrate into target parts of the skin where the substances exert their action at predetermined levels. The permeability of such substances to the skin is determined by the substances', physico-chemical properties. Generally, lipophilic molecules rather than hydrophilic molecules, molecules having low molecular weights rather than those having high molecular weights, and small amounts rather than large amounts of molecules more easily penetrate the skin.

For substances having low permeability to the skin, permeation enhancement may be achieved using specific formulations or techniques so that such substances may exert their effects when applied to the skin. Also, dosages and formulations of substances of interest may be determined based on data obtained from skin permeation studies so as to allow functional products containing the substances to have the strongest efficacy.

The spaces between cells in the stratum corneum are composed of alternate layers of lipids and water, which form multiple lamellar structures. Since the lipid layer has physicochemical properties similar to the plasma membrane (FIG. 1), most hydrophilic molecules, such as adenosine or vitamin c, rarely penetrate the intercellular spaces in the stratum corneum. Delivery of active substances through the skin is mainly carried out directly through skin cells, through the intercellular spaces, or through sweat ducts and hair follicles. Penetration is typically expected to occur via sweat ducts and hair follicles, but only about 1% of active substances substantially permeate via this route. In fact, most active substances permeate through the intercellular spaces in the stratum corneum. Since the intercellular spaces are 30 to 90 ran thick, molecules of interest must have a size smaller than the thickness of the intercellular spaces so as to penetrate the intercellular spaces. For this reason, a variety of techniques for manufacturing small capsules or micelles less than 100 nm in size have been developed in cosmetic industries. Also, such skin penetration techniques involve the use of various agents capable of enlarging the intercellular space of the stratum corneum, such as alcohol or isopropyl alcohol, the employment of liposomes that are nanoscale capsules having a structure similar to the plasma membrane, and the use of artificial nanocapsules or nanotubes prepared using nanocarbon materials. However, since the skin is a complicated structure that possesses numerous nerves and skin cells, it develops irritations in most cases and inflammations in severe cases when contacting artificial chemicals. Thus, artificial agents enlarging the intercellular spaces have limited applications, and thus, may be used only when penetration through the skin is required despite side effects. Also, artificial nanoscale constructs are problematic in practical applications because they are prepared using artificial carbon materials or other types of artificial materials, and because there is presently not sufficient data to predict their safety upon administration into the body. Thus, liposomes, which have a structure almost identical to the plasma membrane, are most beneficial for permeating active substances into the skin (PSIT Vol. 3, No. 12, 2000, 417-425).

An emulsion composition for cosmetic application is prepared by homogenizing a non-ionic surfactant having relatively low irritation, aqueous- and oil-phase components, and a water-soluble polymer as a thickening agent for increasing stability, along with an active substance, using a homo mixer. The content of each ingredient is suitably adjusted according to the desired viscosity or desired outcome of the application. However, when an emulsion composition is prepared by emulsion using a homomixer, it is difficult to make fine emulsion particles less than 2 μm, and such an emulsion composition is difficult to apply to emulsion products having low viscosity.

Typically, liposomes are well known to be useful for application to the skin particularly when hydrophilic molecules have to penetrate through the skin. However, there is rarely information for the relationship between the size of liposomes and physicochemical properties of molecules to be transported into the skin. In fact, since the spaces between cells in the stratum corneum are very narrow (about 30 to 60 nm) (Journal of controlled release, 32, 1994, 249), it has been predominantly thought that only liposomes 100 nm or less in size can pass through the intercellular spaces. For example, Korean Pat. Registration No. 10-422763 discloses a method of preparing liposomes or nanoemulsions having a size of about 40 to 60 nm, which is the same as the size of the intercellular spaces. However, according to a recent report (J. of Controlled Release 59, 1999, 87-97), liposomes less than 100 nm in size have substantial difficulty arriving at the dermal layer because they are fused with the plasma membrane due to tension of cells when they pass through the stratum corneum, and larger liposomes (500 to 1500 nm), which are likely not to penetrate the skin, can be transported to the dermal layer. The principle of skin permeation of liposomes is not completely understood, but, unlike other micelle structures, liposomes are considered to be able to penetrate through narrow spaces due to their water balloon-like flexible structure (FIG. 2). In this regard, the present inventors intended to develop a method of easily preparing liposomes, which easily penetrate into the skin, are larger than conventional unilamellar liposomes, and are stable.

Liposomes may be prepared by various methods according to desired liposome structures, including preparation methods for unilamellar liposomes, multilamellar liposomes and multiple liquid-crystalline liposomes.

Most commercial liposomes are unilamellar liposomes about 100 nm or less in size. These small liposomes are uniform in size so that they are thermodynamically stable and have high formulation stability. Also, since the unilamellar liposomes provide a good feeling and good characteristics, they were successfully commercialized based on their ability to permeate the intercellular spaces in the stratum corneum. For example, Korean Pat. Laid-open Publication No. 10-2004-12113 describes unilamellar liposomes capable of enhancing transdermal absorption of a physiologically active substance, which are manufactured by preparing uniform emulsion particles as fine as about 100 nm through emulsification using hydrogenated lecithin as an emulsifying agent and a high-pressure homogenizer under high pressure.

The preparation method for multiple liquid-crystalline liposomes, which is not generally used, is described in detail in Korean Pat. Registration No. 10-0222000. This Korean patent does not employ a high-pressure homogenizer, and defines the term “multiple liquid-crystalline” as the form in which a liquid crystalline phase surrounds outer and inner leaflets of the membrane of liposomes while maintaining advantages of liposomes and liquid crystals. However, the Korean patent only provides photographs impossible to use to estimate size as evidence of the successful preparation of such liposomes without data for specific physicochemical properties, skin permeability or mean size of the liposomes. There is no evidence that the liposomes shown in photographs are true liposomes. The liposomes shown in the photographs are considered to be oil-in-water emulsions in a liquid-crystalline state, which are generated by non-ionic surfactant used in the preparation of multiple liquid-crystalline. The inventors of the Korean patent described that polyoxyethylene stearate, as an emulsifier for forming a liquid crystalline structure, strengthens inner and outer leaflets of the membrane of liposomes. However, since it is well known in the art that most liposome structures are easily destroyed by any surfactant, a surfactant used for strengthening liposomes is considered to destroy liposome structure, leading to the formation of oil-in-water emulsions in a liquid crystalline state. Thus, the multiple liquid-crystalline liposomes obtained from the Korean patent are believed to be oil-in-water emulsions in a liquid crystalline state, which are generated by reactions between polyoxyethylene stearate and other additionally used emulsifiers (FIG. 3).

Since multilamellar liposomes are not uniform in size, their preparation is difficult to standardize, thus making it difficult to establish standard preparation techniques for multilamellar liposomes. In particular, due to their varying sizes, they, as time goes by, undergo precipitation, phase separation, and fusion of relatively small liposomes with large liposomes and growth due to such fusion. Liposomes burst when their growth in size reaches some levels while releasing ingredients entrapped therein or lipophilic molecules of the membrane thereof. These released molecules rise to the surface of an aqueous layer and form a thin oil film in an upper part. Due to these problems of multilamellar liposomes and the successful preparation of unilamellar liposomes, multilamellar liposomes have rarely been studied and developed.

Recently, based on a new theory for skin penetration and supporting evidence, many studies have been made to develop multilamellar liposomes.

Multilamellar liposomes can be prepared by a method known in the art. For example, multilamellar liposomes may be prepared by dissolving a lipid composition in an organic solvent, evaporating the solvent to form a lipid layer, and hydrating the lipid layer by ultrasonication. Also, as described in Korean Pat. Registration No. 10-0115076, fine multilamellar vesicles of 0.035 to 2 μm may be prepared with natural lipids and natural emulsifying agents using a high-pressure homogenizer.

In addition, U.S. Pat. No. 4,761,288 discloses a method of preparing multilamellar liposomes by extrusion through a high-pressure homogenizer not under general high-pressure homogenization but under a low pressure of about 500 psi. However, this method provides a complex process because it includes first dissolving phosphatidylcholine in a solvent, evaporating the solvent to form a thin film and adding an aqueous liquid to the lipid film. U.S. Pat. No. 4,485,054 discloses a method of preparing multilamellar liposomes by subjecting a lipid film, formed after emulsification, to ultrasonication so as to form spherical liposomes. The above two methods require specific equipment or specific and stringent conditions for liposome preparation, so that they are difficult to be industrialized.

A high-pressure homogenizer is generally used for the preparation of liposomes. The representative example is a microfluidizer. The microfluidizer is a machine performing emulsification using a high pressure of 200 to 2000 atm, and utilizes, to make emulsified particles having a fine size, the principle of cavitation and turbulence caused by pressure change occurring when emulsified products are discharged under room pressure of 1 atm. This machine facilitates the preparation of nano-sized emulsified micelles. However, technical skill is required to use such a high-pressure homogenizer, and high-pressure homogenization conditions greatly affect the characteristics of liposomes. Due to these drawbacks, the method using a high-pressure homogenizer has technical limitations in industries related to cosmetic products and medicaments.

To solve these problems encountered in the prior art, the present inventors intended to easily prepare multilayered liposomes having high skin permeation and high stability and encapsulating a larger amount of an active substance using only conventional equipment (e.g. general homo mixer). The present inventors found that multilayered liposomes, which have a larger size than the size of the intercellular spaces in the stratum corneum, have excellent skin permeation, encapsulate a large amount of a physiologically active substance and are stable, can be prepared not using phosphatidylcholine alone as an emulsifying agent but using a mixture of oil-phase components having a composition similar to that of the plasma membrane in a specific ratio, and using a general homogenizer operating at low speed instead of a high-pressure homogenizer, thereby leading to the present invention.

### DISCLOSURE OF THE INVENTION

Accordingly, the present invention has been made keeping in mind the above problems occurring in the prior art, and an object of the present invention is to provide a multilayered liposome for transdermal absorption, which has excellent skin permeability, encapsulates a large amount of a physiologically active substance, enhances the stability of the active ingredient, and is prepared by a simple manufacturing process and is thus produced at low cost.

Another object of the present invention is to provide a method of preparing the multilayered liposome for transdermal absorption.

A further object of the present invention is to provide a composition for transdermal absorption, comprising a physiologically active substance, which is encapsulated in the multilayered liposome for transdermal absorption.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic presentation in which a physiologically active substance penetrates through the intercellular spaces in the stratum corneum into the dermal layer of the skin;

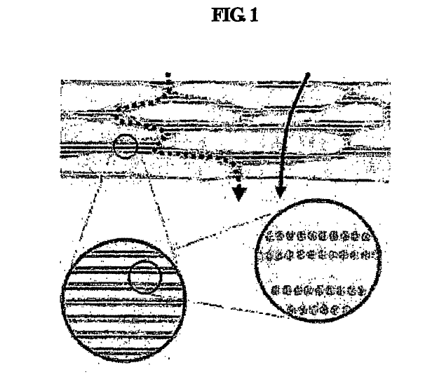

FIG. 2 is a schematic presentation in which a physiologically active substance in the state of being entrapped in liposomes penetrates into the dermal layer of the skin;

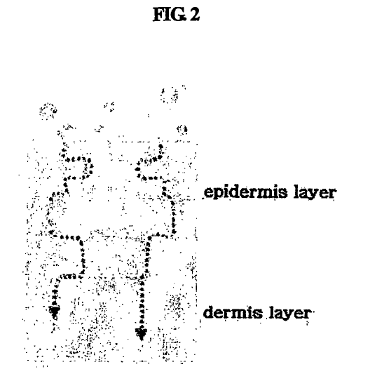

FIG. 3 is a microscopic photograph of oil-in-water emulsions at a liquid crystalline phase; and

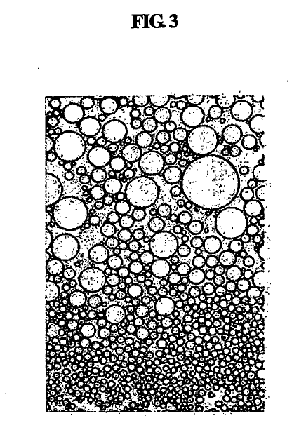

FIG. 4 is a microscopic photograph of multilayered liposomes prepared according to the present invention.

### BEST MODE FOR CARRYING OUT THE INVENTION

In one aspect, the present invention relates to a multilayered liposome for transdermal absorption which is capable of entrapping a physiologically active substance, wherein the liposome is prepared using a mixture of oil-phase components comprising squalane, sterols, ceramides, neutral lipids or oils, fatty acids and lecithins and is 200 to 5000 nm in particle size.

“Liposomes” generally refer to lipid bilayers that are highly-ordered flattened micelles or closed membranes formed when lipids having both hydrophilic and hydrophobic regions are dispersed in water so that the lipids are in equilibrium with water. “Multilayered liposomes (also called multilamellar)”, as used herein, indicates multiple concentric spheres of lipid bilayers, where water layers alternate with lipid bilayers in the fashion: (water layer-lipid bilayer) (water layer-lipid bilayer).

Since the liposomes of the present invention are created not in unilamellar form but in multilamellar form in which several bilayers are stacked, they greatly increase in volume and thus may entrap 100 to 1000 times greater amounts of an active substance than unilamellar liposomes. Also, in comparison with unilamellar liposomes, since the multilayered liposomes of the present invention do not expose their oil-phase components directly to an external water layer except when the oil-phase components are present in the outermost shell, substances in their internal oil or aqueous phases are less influenced by oxidative stress, light, metal ions and several other external environmental factors. Therefore, multilayered liposomes are particularly beneficial for entrapping unstable substances.

In a preferred aspect, the multilayered liposome of the present invention is prepared using a mixture of oil-phase components, which comprises 0.1 to 15.0 wt % of squalane, 0.1 to 10.0 wt % of sterols, 0.1 to 15 wt % of ceramides, 0.1 to 30.0 wt % of neutral lipids or oils, 0.1 to 30.0 wt % of fatty acids and 0.1 to 10.0 wt % of lecithins. In a more preferred aspect, the multilayered liposome of the present invention is prepared using a mixture of oil-phase components, which comprises 0.1 to 10.0 wt % of squalane, 0.1 to 5.0 wt % of sterols, 0.1 to 10 wt % of ceramides, 0.1 to 20.0 wt % of neutral lipids or oils, 0.1 to 20.0 wt % of fatty acids and 0.1 to 5.0 wt % of lecithins. In the most preferred aspect, the multilayered liposome of the present invention is prepared using a mixture of oil-phase components, which comprises 0.1 to 5.0 wt % of squalane, 0.1 to 2.5 wt % of sterols, 0.1 to 5.0 wt % of ceramides, 0.1 to 10.0 wt % of neutral lipids or oils, 0.1 to 10.0 wt % of fatty acids and 0.1 to 2.5 wt % of lecithins.

Squalane, used in the preparation of the multilayered liposomes of the present invention, is an oil material that is highly stable and chemically inert. Squalane is contained in liver oil of shark, the livers of various animals and human sebum, and is also found in olive oil, sesame oil, rice bran oil and yeast. Squalane (C₃₀H₆_{²}), which is obtained by adding hydrogen to squalene (C₃₀H₅₀) in the -presence of a nickel catalyst, is compatible with skin and improves transdermal absorption, and thus may be beneficially used. In the preparation of the multilayered liposomes of the present invention, animal or vegetable squalane or derivatives thereof may be used alone or in combinations of two or more.

Sterols are used as softening agents, emulsifying agents and emulsion stabilizers because they have good skin permeability and cause less irritation, and they stabilize vesicles. Animal sterols and vegetable sterols are all available, and are exemplified by cholesterol, campesterol, stigmasterol, β-sitosterol and fucosterol. In the preparation of the multilayered liposomes of the present invention, sterols may be used alone or in combinations of two or more.

Ceramides are a member of sphingolipids, and are prepared from various fatty acids such as sphingosine or linoleic acid. A ceramide consists of a fatty acid linked to the amino group of sphingosine, a long-chain base, through an amide bond. Ceramides primarily have a barrier function, and also function to bind to water and control immune responses. All types of ceramides, derived from animals and plants, are available, and are exemplified by phytosphingosine and ceramide III. In the preparation of the multilayered liposomes of the present invention, ceramides may be used alone or in combinations of two or more.

Neutral lipids indicate triglycerides, and oils include all types of vegetable oils and animal oils. Examples of vegetable oils include olive oil, camellia oil, rice bran oil and macadamia nut oil, and examples of animal oils include tallow, lard and ostrich oil. In the preparation of the multilayered liposomes of the present invention, neutral lipids, vegetable oils and animal oils may be used alone or in combinations of two or more.

Available fatty acids include all types of fatty acids used as cosmetic or medical raw materials. Fatty acids having β to 20 carbons are preferred, which may be straight or branched. Examples of preferred fatty acids include, but are not limited to, stearic acid, oleic acid, linoleic acid, palmitic acid, linolenic acid and myristic acid.

Lecithin, which is a phospholipid, has a hydrophilic moiety, including phosphoric acid and choline, on one carbon of the glycerol backbone and hydrophobic acyl groups on two other carbons of the glycerol backone. Due to this nature, lecithin may be contained in both aqueous and oil phases. Preferably, lecithin has a fatty acid chain of 12 to 24 carbons and contains 20% or more phosphatidylcholine. In the preparation of the multilayered liposomes of the present invention, derivatives of lecithin may be preferably used, which are exemplified by hydrogenated lecithin formed by saturation of unsaturated double bonds of lecithin, lysolecithin prepared by partial hydrolysis of fatty acid chains of a phospholipid, and hydroxylated lecithin prepared by introducing a hydroxyl group into lecithin. The aforementioned lecithins and derivatives thereof may be derived from animals or plants. In the preparation of the multilayered liposomes of the present invention, the plant- or animal-derived lecithins or derivatives thereof may be used alone or in combinations of two or more.

The multilayered liposomes of the present invention may further include an antiseptic, an antioxidant, a stabilizer, a thickener, and the like to improve their stability. All synthetic and natural antiseptics are available, and their mixtures are also available. An antiseptic is typically used in an amount of 0.01% to 20%. Available antioxidants include BHT, erysorbate, tocopherol, astaxanthin, vegetable flavonoid, and derivatives thereof, and further include various plant-derived antioxidizing substances. A stabilizer may be added to constructed liposomes to stabilize liposome structure, and is exemplified by polyols and sugars. Examples of polyols include, but are not limited to, butylene glycol, polyethylene glycol, propylene glycol, dipropylene glycol and ethyl carbitol. Examples of sugars include, but are not limited to, trehalose, sucrose, mannitol, sorbitol and chitosan. Monosaccharides, oligosaccharides, high molecular weight starches, and the like are also available. A thickener used for improving the dispersion stability of constructed liposomes in water includes various natural thickeners, and acrylamides and synthetic polymeric thickeners are available. Examples of thickeners include, but are not limited to, natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as polyacrylic acid, polyacrylamide, polyvinylpyrollidone and polyvinylalcohol, and copolymers thereof, and cross-linked materials.

The multilayered liposomes of the present invention entrap a physiologically active substance. The physiologically active substance includes all substances capable of being entrapped in liposomes and enhancing physiological functions, and is not particularly limited. Examples of the physiologically active substance include. proteins, peptides, nucleic acids, synthetic compounds, natural extracts, sugars, vitamins and inorganic materials, and may be naturally isolated, chemically synthesized or recombinantly produced. As an illustrative example, the physiologically active substance includes toxins, enzymes, hormones, neurotransmitters, immunoglobulins and polysaccharides. As another illustrative example, the physiologically active substance includes, but is not limited to, immunoregulators, antibiotics, antitumor agents, anti-inflammatory agents, antipyretics, analgesics, antiedemic agents, antitussive expectorants, sedatives, muscle relaxants, antiepileptic agents, antiulcerants, antidepressants, antiallergic agents, cardiac stimulants, antiarrhythmic agents, vasodilators, hypotensive agents, antidiabetic agents, homeostatic agents, hormone agents, antioxidants, hair growth promoters, antibacterial agents, skin whitening agents, collagen synthesis stimulators, wrinkle removing/relieving agents, skin barrier strengthening agents, skin moisture enhancers, and cosmetic agents

The multilayered liposomes of the present invention preferably consist of 3 to 20 membranes, and contain a hydrophobic active ingredient present in the membranes, and a hydrophilic active ingredient in the inner core region of the liposomes and in the spaces between the membranes. Also, in the present liposomes, bipolar substances are present in a form spanning a membrane while exposing their polar groups to the inner core region or a space between membranes. These multilayered liposomes of the present invention have a particle size ranging from 200 nm to 5000 nm, preferably 200 nm to 3000 nm, and more preferably 800 nm to 1000 nm.

In another aspect, the present invention relates to a composition for transdermal absorption, comprising a physiologically active substance, which is encapsulated in the multilayered liposome of the present invention.

The application of the present composition is not particularly limited. The composition has various applications, which include basic cosmetic products, such as skin softeners, skin nutrition lotions, creams, packs, gels and patches; coloring cosmetic products, such as lipsticks, make-up bases and foundation; cleaning preparations, such as shampoos, rinses and body cleansers; oral compositions, such as toothpastes and oral cleaners; hair compositions, such as hair styling products, e.g., hair tonics, gels and mousses, hair growth promoters, and hair coloring products; and medicaments and medical supplies, such as lotions, ointments, gels, creams, patches and sprays.

In a further aspect, the present invention relates to a method of preparing multilayered liposomes for transdermal absorption, comprising (a) dissolving oil-phase components, comprising squalane, sterols, ceramides, neutral lipids or oils, fatty acids and lecithins, at 50° C. to 75° C., (b) dissolving aqueous-phase components at 50° C. to 75° C., (c) mixing the components dissolved at steps (a) and (b) and agitating a resulting mixture at 500 to 9000 rpm (revolutions per minute) to form multilayered liposomes having a particle size of 200 to 5000 nm.

Oil-phase components used in the preparation of the multilayered liposomes of the present invention are as described above, and are present in an amount of 1 to 30 wt %, and preferably 1 to 5 wt %, based on the total weight of the composition.

In addition to water, aqueous-phase components may include polyols, such as butylene glycol and propylene glycol. Also, the aqueous-phase components may further include substances having antioxidizing activity, such as hydrophilic vegetable flavonoid or a rosemary extract.

When the multilayered liposomes of the present invention are prepared, oil-phase components and aqueous-phase components are individually mixed and dissolved in a dissolving tank using a homo mixer or a paddle mixer. The oil-phase components are dissolved in an organic solvent, preferably an alcohol such as methanol, ethanol, n-propanol, isopropanol or butanol, and more preferably ethanol. When the oil-phase components are dissolved in an alcohol, they are easily dissolved at a relatively low temperature of about 60° C. and may be completely dissolved.

A mixture of oil-phase components, prepared in a separate agitator, is added to a mixture of aqueous-phase components and agitated at 500 to 9000 rpm, preferably 2000 to 4000 rpm, and more preferably 3000 rpm, for 1 to 30 min, preferably 3 to 10 min, and more preferably 4 to β min. This emulsification method is commonly used in the art to make oil-in-water emulsions, and thus, learning a particular technique is not required. The emulsion by agitation may be achieved using a variety of emulsifying dispersion means widely used in the art, such as propeller-type mixers, Disper, homo mixers, homogenizers, colloidal mills, and ultrasonic emulsifying means. In particular, the homo mixer is an agitation apparatus that is generally used for homogeneous mixing of medicaments, cosmetic products and foods. Liposomes may be prepared in a suitable size by controlling the agitation speed and time.

As noted above, the present invention employs a general low-speed homogenizer while not using a high-pressure homogenizer for preparing multilayered liposomes, thereby facilitating the preparation of multilayered liposomes in industries manufacturing medical raw materials or cosmetic industries.

The multilayered liposomes prepared according to the present invention are fine uniform particles, which have a particle size, preferably ranging from 200 to 5000 ran, more preferably ranging from 200 to 1500 ran, and even more preferably 800 to 1000 ran, and a viscosity of 1 to 5000 cps.

The physiologically active substance, contained in the present multilayered liposomes, is dissolved in the oil-phase components if it is hydrophobic, or in the aqueous-phase components if it is hydrophilic. When the physiologically active substance is thermally unstable, it may be added to an emulsified product and agitated at 45° C. or lower in order to be introduced into prepared liposomes.

In addition, a secondary process may be performed in order to prepare the liposomes obtained in the present invention in a more uniform form. The primarily prepared multilayered liposomes may pass, for example, through a high-pressure homogenizer or a microfludizer under high pressure to obtain more uniformly multilayered liposomes.

The multilayered liposomes of the present invention, formed from a mixture of oil-phase components and a mixture of aqueous-phase components, which are prepared in separate agitators according to the present invention, using a general homo mixer, were found to have excellent stability that maintains the particle size of liposomes even after twelve months. Also, with respect to transdermal absorption efficiency, the present multilayered liposomes displayed skin permeation rates or amounts similar to those of conventional multilayered liposomes prepared using a high-pressure homogenizer. That is, without using an expensive apparatus such as a high-pressure homogenizer, the present invention provides multilayered liposomes, which are transdermally absorbed in levels similar to conventional multilamellar liposomes prepared using such an expensive apparatus. Also, irritation tests on human skin demonstrated that the present multilayered liposomes are safe to the human body without irritation.

The present method of preparing liposomes provides a simple and cost-effective process and makes it possible to make multilayered liposomes, biocapsules or particles that enhance the transdermal absorption of a physiologically active substance entrapped therein. Thus, the present method is applicable to cosmetic products, foods and medicaments for healing and the alleviation of wounds, skin care, and the like.

A better understanding of the present invention may be obtained through the following examples which are set forth to illustrate, but are not to be construed as the limit of the present invention.

### EXAMPLE 1

### Preparation of Multilayered Liposomes Using a General Homo Mixer

Multilayered liposomes were prepared According to the present method, which is characterized by forming multilayered liposomes using a general low-speed homogenizer without a specific machine such as a high-pressure homogenizer, for example, a microfludizer. In detail, multilayered liposomes were prepared according to the following procedure.

1) Adenosine was added to water warmed to 50° C. in a dissolving tank and agitated using a paddle mixer to be completely dissolved.

2) Oil-phase components listed in Table 1, below, were added to a separate dissolving tank and heated to 70° C. to 75° C. to be completely dissolved.

3) The oil-phase components of step 2 were added to the dissolving tank of step 1 and mixed for 5 min at 3000 rpm using a homo mixer to form liposomes.

4) The mixture of step 3 was cooled to 45° C. and supplemented with an antioxidant, a thickener, an antiseptic and the like to increase the storage stability of liposomes.

The amount of ingredients used in the preparation of the multilayered liposomes of the present invention is given in Table 1, below.

TABLE 1
Composition of the present mulilayered liposomes

| | | wt % | | |
|---|---|---|---|---|
| | Ingredient | T. E. 1 | T. E. 2 | T. E. 3 |
| Oil phase | Squalane | 3.00 | 3.00 | 3.00 |
| | Ceramide | 1.00 | 1.00 | 1.00 |
| | Vegetable sterol | 2.00 | — | 2.00 |
| | Cholesterol | — | 2.00 | — |
| | Camellia oil | 5.00 | 5.00 | 5.00 |
| | Stearic acid | 1.00 | 1.00 | 1.00 |
| | Ethanol | 10.00 | 10.00 | 10.00 |
| | Lecithin | 2.50 | — | 1.00 |
| | Hydrogenated | — | 2.50 | 1.50 |
| | lecithin | | | |
| | Tocopherylacetate | 0.3 | 0.3 | 0.3 |
| Aqueous | Adenosine | 0.04 | 0.04 | 0.04 |
| phase | Purified water | Up to 100 | Up to 100 | Up to 100 |
| Additives | Naturotics | 2.00 | 2.00 | 2.00 |
| | (natural antiseptic) | | | |
| | Xanthan gum | Suitable | Suitable | Suitable |
| | | amount | amount | amount |
| | Vegetable polyphenol | Suitable | Suitable | Suitable |
| | | amount | amount | amount |
| | Sorbitol | Suitable | Suitable | Suitable |
| | | amount | amount | amount |

### COMPARATIVE EXAMPLE 1

### Preparation of Nano-Sized Liposomes Using High-Pressure Homogenizer

Nano-sized liposomes were prepared according to the method described in Korean Pat. Laid-open Publication No. 10-2004-0012113, except that adenosine instead of albutin used in the patent publication was used as an active substance functioning as an indicator of transdermal drug delivery efficiency of liposomes in the present invention.

| Ingredients | % |
|---|---|
| Vegetable squalane | 6.0 |
| Phenyl trimethicone | 3.0 |
| Cyclopentasiloxane | 2.0 |
| Meadowfoam seed oil | 0.1 |
| Tocopherylacetate | 0.1 |
| Glycerin | 4.0 |
| Adenosine | 0.04 |
| Trisodium EDTA | 0.01 |
| Dipropylene glycol | 5.0 |
| Hydrogenated lecithin | 1.0 |
| Ethanol | 3.0 |
| Antiseptic | suitable amount |
| Perfume | suitable amount |
| Colorant | suitable amount |
| Purified water | up to 100 ml |

### COMPARATIVE EXAMPLE 2

### Preparation of Liposomes by Thin Film Method Using an Organic Solvent

Liposomes were prepared according to the same procedure as described in Example 1 of U.S. Pat. No. 4,761,288, except that adenosine instead of minoxidil used in the U.S. patent was used as an active ingredient functioning as an indicator of transdermal drug delivery efficiency of liposomes in the present invention.

| Ingredients | | |
|---|---|---|
| Di-alpha dipalmitoyl phosphatidylcholine | 400 | mg |
| Choresterol | 200 | mg |
| Adenosine | 40 | mg |
| Ethanol | 1 | ml |
| Propylene glycol | 0.7 | ml |
| Calcium chloride (8 mM) | 8.3 | ml |

### COMPARATIVE EXAMPLE 3

### Preparation of Multiple Liquid-Crystalline Liposomes Using a General Homo Mixer

Multiple liquid-crystalline liposomes were prepared according to the same procedure as described in Example 1 of Korean Pat. Registration No. 10-0222000 except for the use of adenosine as an active ingredient.

| Ingredients | % |
|---|---|
| Polyoxyethylene stearate | 3.15 |
| Polyoxyethylene dihydroxy stearate | 0.64 |
| Heptamethylnonane | 5.00 |
| Tocopherol acetate | 0.50 |
| High-grade alcohol | 1.50 |
| High-grade fatty acids | 1.50 |
| Microcrystalline lead | 0.50 |
| Fatty acid glyceride | 6.00 |
| Purified water | 10.00 |
| Butylenes glycol | 1.50 |
| Concentrated glycerin | 2.00 |
| Antiseptic | 0.40 |
| Urea | 0.30 |
| Phospholipids | 0.50 |
| Purified water | 56.61 |
| Mixed perfumes | 0.20 |
| Adenosine | 0.04 |
| Silicon oil | 1.00 |

### COMPARATIVE EXAMPLE 4

### Preparation of Multilayered Liposomes Using a High-Pressure Homogenizer

Multilayered liposomes were prepared according to the same method as described by IY Kim et al. in a paper published in a Korean cosmetic journal, 21-1 (vol. 38), 1995, except for the use of adenosine as an active ingredient.

| Ingredients | % |
|---|---|
| Lipids | 15.0-20.0 |
| Propylene glycol | 5.0 |
| Cetylphosphate | 0.5 |
| Purified water | suitable amount |
| Malic acid | 1.0 |
| Tartaric acid | 1.0 |
| Lactic acid | 1.0 |
| Purified water | up to 100 |

### EXAMPLE 2

### Measurement of Liposome Size

The size of liposomes prepared in Example 1 and Comparative Examples (CE.) 1 to 4 was measured. The size of emulsified particles was measured three times for each sample using a particle size analyzer (Model 370, Nicomp, USA). Mean values of the measured results and the results obtained by 600× microscopic observation are given in Table 2, below.

TABLE 2
Liposome size measured using particle size analyzer

| | C.E.1 | C.E.2 | C.E.3 | C.E.4 | T.E.1 | T.E.2 | T.E.3 |
|---|---|---|---|---|---|---|---|
| Particle | 30-200 | 1000-15000 | >3000 | 150-350 | 200-1500 | 200-1500 | 200-1500 |
| size | | | | | | | |
| distribution | | | | | | | |
| Mean | 100 | 4000 | 4000 | 250 | 800 | 1000 | 900 |
| particle | | | | | | | |
| size (nm) | | | | | | | |
| Structure of | Uni- | Multi- | O/W liquid | Mixed | Multi- | Multi- | Multi- |
| liposomes | lamellar | layered | crystalline | (unilamellar | layered | layered | layered |
| | | | | and | | | |
| | | | | multilayered) | | | |

As apparent from the data of Table 2, unilamellar (Comparative Example 1) and multilayered (Comparative Example 4) liposomes, which were prepared using a high-pressure homogenizer, all had a uniform and small size compared to liposomes prepared without using a high-pressure homogenizer. It has been well known in the art that liposome size is affected by pressure and temperature upon passage through a high-pressure homogenizer and passage frequency. When multilayered liposomes were made by forming thin bilayers using an organic solvent and hydrating the bilayers (Comparative Example 2), they greatly varied in size ranging from 1 μm to 15 μm. These multilayered liposomes are thermodynamically very unstable due to their highly irregular size. Also, in this case, relatively small liposome particles approach large liposome particles due to the tension of the large liposomes and were eventually fused thereto. Repeated liposome fusion leads to the growth of liposomes, and when fused liposomes reach a limit point they burst like balloons. Since, as time goes by, the structure of liposomes becomes unstable and liposomes become disrupted, oils gathered with each other in a highly ordered fashion become exposed to an aqueous phase and finally rise to the top of emulsions, resulting in the formation of an oil layer. Thus, although the method of Comparative Example 2 provides a relatively simple process for preparing liposomes, it is considered unsuitable for industrial applications.

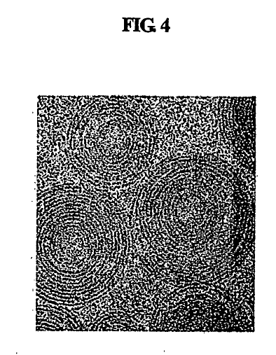

In contrast, in the case of liposomes prepared in Test Examples (T.E.) 1, 2 and 3, particle size distribution was uniform in a range of about 200 to 15000 nm, and mean particle size ranged from about 800 to 1000 nm. Also, microscopic observation revealed that the liposomes all have a multilayered structure of 3 to 20 or more layers (FIG. 4). FIG. 4 is a microscopic photograph of liposomes prepared in Test Example 1.

### EXAMPLE 3

### Evaluation of Stability of Liposomes

The liposomes prepared in Test Example 1 and Comparative Examples 1 to 4 were stored in an incubator at 25° C. under relative humidity of 70%±5 and observed for their stability. The results are summarized in Table 3, below. In Table 3, the particle size of liposomes is expressed as ran.

TABLE 3
Stability of liposomes

| | C.E. 1 | C.E. 2 | C.E. 3 | C.E. 4 | T.E. 1 | T.E. 2 | T.E. 3 |
|---|---|---|---|---|---|---|---|
| Immediately after preparation | 100 | 4000 | 4000 | 250 | 800 | 1000 | 900 |
| After 2 weeks | 100 | 4200 | 4010 | 280 | 800 | 900 | 902 |
| After 1 month | 110 | 6020 | 3988 | 410 | 810 | 1010 | 913 |
| After 3 months | 130 | 4020 | 4207 | 1100 | 805 | 989 | 897 |
| After 12 months | 210 | 6200 | 4023 | 980 | 796 | 1012 | 902 |

As apparent from the data of Table 3, nano-sized liposomes of about 100 ran, prepared in Comparative Example 1, were thermodynamically stable, did not change in size for a storage period of one month and enlarged only about two times after three months. Multiple liquid-crystalline liposomes prepared in Comparative Example 3 were considered not to be liposomes but oil-in-water (O/W) emulsions at a liquid crystalline phase, and were stable in size after twelve months. Multilayered liposomes prepared using an organic solvent in Comparative Example 2 were maintained in size ranging from about 4000 to 6000 nm. Liposomes prepared in Comparative Example 4 greatly changed in size as time passed. The present multilayered liposomes prepared in Test Examples 1 to 3 all maintained their size in a relatively uniform range. In particular, multilayered liposomes prepared in Test Example 1 were found to have the highest structural stability.

### EXAMPLE 4

### Evaluation of the Skin Permeability of Liposomes

Liposomes prepared in Test Example 1 and Comparative Examples 3 and 4 were tested for their skin permeability. The subcutaneous absorption of liposomes was measured in the skin of hairless guinea pigs (Jackson Laboratories) using Franz permeation cells (PermeGear, Inc.). Right before this test, the skin at the abdomen of hairless guinea pigs was collected and cut to a size of 1 cm². The skin pieces were mounted between donor and receptor compartments of Franz permeation cells having an orifice diameter of 0.9 cm, and immobilized with clamps. 0.5 ml of each of the liposomes prepared in Test Example 1 and Comparative Examples 3 and 4 were applied onto the skin surface in the donor compartment. The opposite side of the skin in the receptor compartment was allowed to contact a solvent solution, that is, a 4:1 mixture of purified water and ethanol. The temperature of the entire permeation cell assembly was maintained at actual skin temperature, 32° C. A portion of the solvent was collected at given time points The amount of adenosine permeating the skin was measured, and expressed as subcutaneous absorption per unit applied concentration (μg/cm²/wt %). The results are given in Table 4, below.

Quantitative analysis of adenosine was performed by gas chromatography and high performance liquid chromatography under the following conditions.

[Gas Chromatography]

Injector: split ratio: 1:50

Detector: FID (Flame Ionization Detector)

Column: 30 m DBWAX 0.25 mm LD

Column pressure: 10 psi

Injector temperature: 250° C.

Detector temperature: 250° C.

Oven temperature program

Start: 200° C.

Heating rate: 4° C./min, up to 250° C.

[High Performance Liquid Chromatography]
Equipments: Dionex P530 pump, ASI-100 automated sample injector
Column: Phenomenex Luna 5u (C18 (2)), 150×4.6 mm
Mobile phase: 10 mM KH2PO4:water=92:8 (0-6.5 min)-40:60 (7.5-12 min)-92:8 (12.5-15 min) gradient system
Flow rate: 0.6 ml/min
Detector: UV/Vis Detector UVD 340S 260 nm
Injection volume: 10 μl

Operation time: 25 min

TABLE 4
Subcutaneous absorption (μg/cm²) of adenosine

| | | Elapsed time (hr) | | | | | | | | | | | | | | | |
|---|---|---|---|---|---|---|---|---|---|---|---|---|---|---|---|---|---|
| | | 1 | | | | 2 | | | | 3 | | | | Mean | | | |
| | Liposomes | 0 | 4 | 8 | 12 | 0 | 4 | 8 | 12 | 0 | 4 | 8 | 12 | 0 | 4 | 8 | 12 |
| Cumulative | C.E. 3 | 0 | 5 | 15 | 20 | 0 | 8 | 18 | 23 | 0 | 4 | 14 | 19 | 0 | 5.7 | 15.7 | 20.7 |
| amount | C.E. 4 | 0 | 76 | 160 | 175 | 0 | 87 | 168 | 178 | 0 | 97 | 181 | 193 | 0 | 86.7 | 169.7 | 182 |
| | T.E. 1 | 0 | 66 | 171 | 180 | 0 | 48 | 156 | 192 | 0 | 69 | 164 | 179 | 0 | 61 | 163.7 | 183.7 |

As apparent from the data of Table 4, the O/W liquid crystals of Comparative Example 3, identified to have a general liquid crystalline structure, rarely transported adenosine into the skin. Compared to Comparative Example 3, the multilayered liposomes prepared using a microfluidizer in Comparative Example 4 effectively delivered adenosine into the skin in a time-dependent manner. The multilayered liposomes of the present invention showed permeation rates or amounts of adenosine through the skin almost identical to those of liposomes of Comparative Example 4. These results indicates that, when liposomes are prepared according to the present method using a general low-speed homo mixer, they can transdermally deliver physiologically active substances with efficiency similar to that of conventional liposomes prepared using expensive equipment.

### EXAMPLE 5

### Evaluation of Safety of Liposomes to a Body

A patch test was carried out to determine whether liposomes cause irritation on human skin. In this test, the degree of irritation was determined, and it was also assessed whether developed irritation could be relieved by some relieving agents. The test was performed by Dermapro Co. Ltd., Korea, which is a research organization specializing in the dermatological testing of cosmetics. Thirty human subjects were patch-tested, and test results were excluded when samples were deemed to be unsuitable as cosmetic materials. A closed patch test was performed while patches were applied on suitable areas of the body, such as the upper region of the upper part of the back (except for central middle areas) or the forearm. After 48 hrs, patches were detached from applied skin. After transient edema due to the patch removal disappeared, the occurrence of irritation was determined under the supervision of dermatology specialists having five or more years' experience in the dermatology field.

In detail, the patch test was performed according to the following procedure. Thirty human subjects were patch-tested using a Finn Chamber (5 mm in diameter), and patches were applied onto the normal skin (the back or the forearm) of the subjects. The small amount of each sample was added to a Finn Chamber, and the Finn Chamber was mounted on a Scanpor tape and attached onto the subject's skin. After two days, the patch was removed, and the applied area of the skin was observed for the occurrence of irritation. After another two days, the reading of the same area of the skin was taken again. The patch test results were interpreted according to the interpretation standard, below, which is recommended by the International Contact Dermatitis Research Group. The subjects were prohibited from taking any antihistamines during a one-week period from three days before the test until the reading was finished

[The Interpretation Standard Recommended by the International Contact Dermatitis Research Group]

±doubtful reaction

1+=weak positive reaction (non-vesicular)

2+=strong positive reaction (vesicular)

3+=extreme positive reaction

4+=irritant reaction

As a result of the patch test for the safety of liposomes to the human body, the multilayered liposomes of the present invention did not cause irritation even after 72 hrs, indicating that they have high safety (Table 5)

TABLE 5
Test for safety of liposomes to a human body

| | | 48 h after patch | | | | | 72 h after patch | | | | | Irritant | | |
|---|---|---|---|---|---|---|---|---|---|---|---|---|---|---|
| Sample | Tested | application | | | | | application | | | | | reaction (n = 30) | | |
| No. | liposomes | ± | 1+ | 2+ | 3+ | 4+ | ± | 1+ | 2+ | 3+ | 4+ | 48 h | 72 h | Mean |
| 1 | C.E. 1 | 2 | — | — | — | — | — | — | — | — | — | 0.8 | 0 | 0.4 |
| 2 | C.E. 2 | 2 | — | — | — | — | — | — | — | — | — | 0.8 | 0 | 0.4 |
| 3 | C.E. 3 | 2 | — | — | — | — | — | — | — | — | — | 0.8 | 0 | 0.4 |
| 4 | C.E. 4 | 1 | — | — | — | — | — | — | — | — | — | 0.4 | 0 | 0.2 |
| 5 | T.E. 1 | — | — | — | — | — | — | — | — | — | — | 0 | 0 | 0 |
| 6 | T.E. 2 | — | — | — | — | — | — | — | — | — | — | 0 | 0 | 0 |
| 7 | T.E. 3 | — | — | — | — | — | — | — | — | — | — | 0 | 0 | 0 |

Industrial Applicability

As described hereinbefore, the multilayered liposomes of the present invention entrap a larger amount of an active ingredient and are structurally stable when entrapping the active ingredient, compared to unilamellar liposomes. Also, the present multilayered liposomes are prepared by a simple and cost-effective process not using a high-pressure homogenizer but using a general homo mixer. Further, since the present multilayered liposomes are prepared in a larger size than the intercellular spaces in the stratum corneum, they by-pass the tension of surrounding cells when passing through the intercellular spaces and are thus able to penetrate into the dermal layer, compared to nano-sized unilamellar liposomes. Therefore, the present multilayered liposomes are useful for enhancing the transdermal absorption of physiologically active substances.

1. A multilayered liposome for transdermal absorption which is capable of entrapping a physiologically active substance, wherein the liposome is prepared using a mixture of oil-phase components comprising 0.1 to 10.0 wt % of squalane, 0.1 to 5.0 wt % of sterols, 0.1 to 10 wt % of ceramides, 0.1 to 20.0 wt % of neutral lipids or oils, 0.1 to 20.0 wt % of fatty acids and 0.1 to 5.0 wt % of lecithins, based on the total weight of the liposome, and is 200 to 5000 nm in particle size.

2. The multilayered liposome according to claim 1, wherein the particle size ranges from 200 to 1500 nm.

3. A method of preparing multilayered liposomes for transdermal absorption, comprising:

4. The method according to claim 3, further comprising secondarily disrupting and mixing the multilayered liposomes by passing the multilayered liposomes through a high-pressure homogenizer.

5. A multilayered liposome for transdermal absorption, prepared according to the method of claim 3.

6. A composition for transdermal absorption, comprising the multilayered liposome of claim 1 or 8 entrapping a physiologically active substance.

7. The composition according to claim 9, wherein the physiologically active substance is selected from among proteins, peptides, nucleic acids, natural extracts, synthetic compounds, sugars, vitamins and inorganic materials.

8. A composition for transdermal absorption, comprising the multilayered liposome of claim 8 entrapping a physiologically active substance.
